(19) United States
(12) Patent Application Publication
(10) Pub. No.: US 2013/0249968 A1
(43) Pub. Date: Sep. 26, 2013

(54) FUSED POLYCYCLIC COMPOUND AND ORGANIC LIGHT EMITTING DEVICE USING THE SAME

(71) Applicants: Naoki Yamada, Inagi-shi (JP); Jun Kamatani, Tokyo (JP); Akihito Saitoh, Gotemba-shi (JP)

(72) Inventors: Naoki Yamada, Inagi-shi (JP); Jun Kamatani, Tokyo (JP); Akihito Saitoh, Gotemba-shi (JP)

(73) Assignee: CANON KABUSHIKI KAISHA, Tokyo (JP)

(21) Appl. No.: 13/990,383

(22) PCT Filed: Nov. 18, 2011
(86) PCT No.: PCT/JP2011/077268

§ 371 Date: May 29, 2013

(30) Foreign Application Priority Data
Dec. 24, 2010 (JP) 2010-286970

(51) Int. Cl. H01L 51/00; G09G 3/32; H01L 27/32

(52) U.S. Cl. CPC H01L 51/0074; H01L 27/3244; G09G 3/3208
USPC 345/690; 257/40; 549/43

(57) ABSTRACT

Provided is an organic light emitting device having high emission efficiency and a low driving voltage. The organic light emitting device includes an anode, a cathode, and an organic compound layer disposed between the anode and the cathode, in which the organic compound layer includes a fused polycyclic compound represented by any one of the following general formulae [1] to [4].

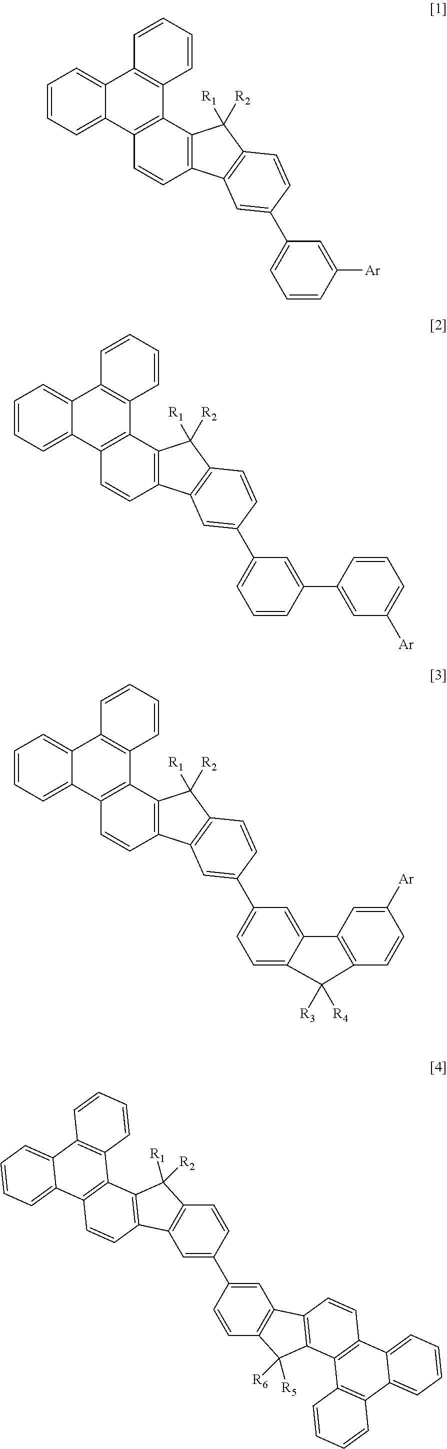

### TECHNICAL FIELD

The present invention relates to a fused polycyclic compound and an organic light emitting device using the same.

### BACKGROUND ART

An organic light emitting device is an electronic element including an anode, a cathode, and an organic compound layer disposed between both the electrodes. Holes and electrons to be injected from the respective electrodes recombine with each other in the organic compound layer, in particular, a light emitting layer. When excitons generated by the recombination return to the ground state, the organic light emitting device emits light.

Recent advances in the organic light emitting device are remarkable, and have resulted in the following features, for example. That is, the organic light emitting device has a low driving voltage, a variety of emission wavelengths, and high-speed responsiveness, and allows a light emitting device to be reduced in thickness and weight.

Meanwhile, the organic light emitting device is broadly classified into a fluorescent light emitting device and a phosphorescent light emitting device depending on the kind of excitons involved in emission. In particular, the phosphorescent light emitting device is an electronic element including a phosphorescent light emitting material in an organic compound layer, specifically a light emitting layer, which constructs the organic light emitting device, in which triplet excitons are involved in emission. Here, the phosphorescent light emitting material is excited to the triplet state through the recombination of holes and electrons, and emits phosphorescent light when returning to the ground state. Thus, the phosphorescent light emitting device is an organic light emitting device which provides emission derived from the triplet excitons.

Meanwhile, the phosphorescent light emitting device has attracted attention in recent years because the internal quantum efficiency of the phosphorescent light emitting device is four times as large as the internal quantum efficiency of the fluorescent light emitting device in theory. However, in the phosphorescent light emitting device, there is a room for further improvement in emission efficiency.

Meanwhile, there are various proposals concerning materials to be used in the phosphorescent light emitting device. For example, there are proposals concerning compounds having the following partial structures disclosed in Journal of Organic Chemistry 2006, 71, 6822-6828 and Japanese Patent Application Laid-Open No. 2008-290991 (corresponding PCT Number: WO2008146825A1).

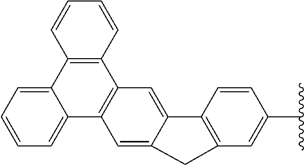 [SMILES: *c1ccc2c(c1)Cc1cc3c4ccccc4c4ccccc4c3cc1-2]

### SUMMARY OF INVENTION

The present invention has been made in order to solve the above-mentioned problems. An object of the present invention is to provide an organic light emitting device having high emission efficiency and a low driving voltage.

A fused polycyclic compound of the present invention is represented by any one of the following general formulae [1] to [4]:

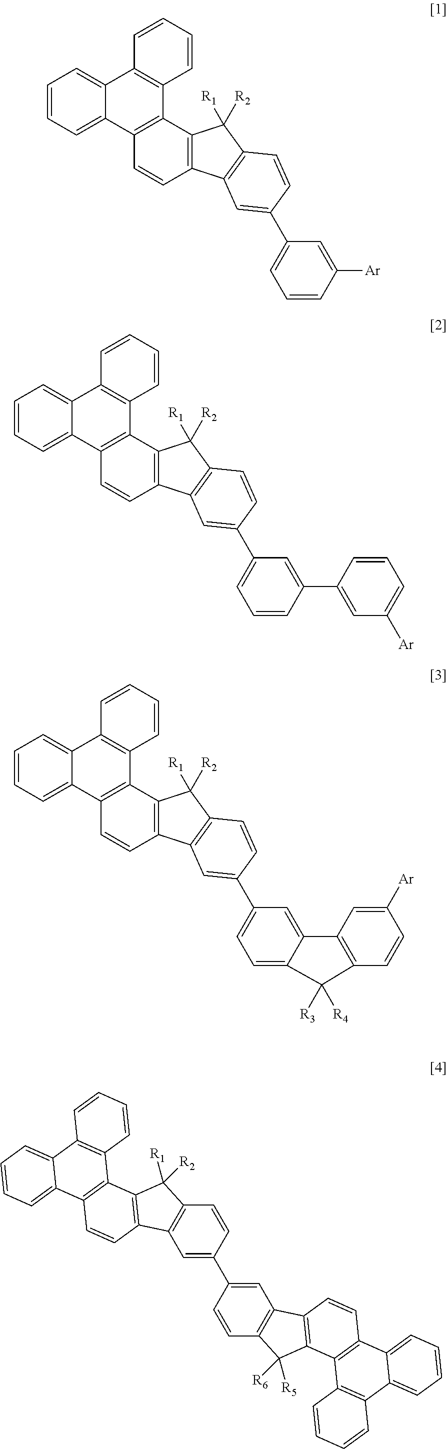

(in the formulae [1] to [4], Ar represents one of a substituted or unsubstituted phenyl group, a substituted or unsubstituted dibenzothiophenyl group, a substituted or unsubstituted phenanthryl group, a substituted or unsubstituted fluorenyl group, a substituted or unsubstituted triphenylenyl group, and a substituted or unsubstituted naphthyl group, and R₁ to R₆ each represent one of a hydrogen atom and an alkyl group having 1 or more to 4 or less carbon atoms; in the formulae [1] to [4], R₁ and R₂ may be identical to or different from each other; in the formula [3], R₃ and R₄ may be identical to or different from each other; and in the formula [4], R₅ and R₆ may be identical to or different from each other).

According to the present invention, it is possible to provide the organic light emitting device having high emission efficiency and a low driving voltage.

### BRIEF DESCRIPTION OF DRAWINGS

FIG. 1 is a cross-sectional schematic diagram illustrating an example of a display apparatus including an organic light emitting device of the present invention and a TFT element as an example of a switching element electrically connected to the organic light emitting device.

### DESCRIPTION OF EMBODIMENTS

First, a fused polycyclic compound of the present invention is described. The fused polycyclic compound of the present invention is a compound represented by any one of the following general formulae [1] to [4].

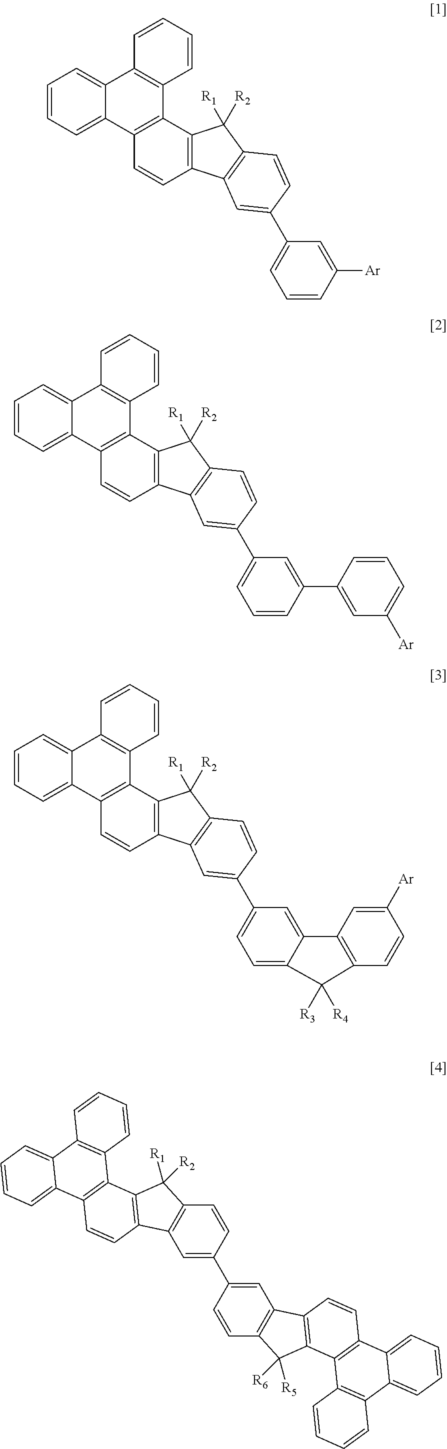

In the formulae [1] to [4], Ar represents one of a substituted or unsubstituted phenyl group, a substituted or unsubstituted dibenzothiophenyl group, a substituted or unsubstituted phenanthryl group, a substituted or unsubstituted fluorenyl group, a substituted or unsubstituted triphenylenyl group, and a substituted or unsubstituted naphthyl group.

A substituent which may be possessed by each of the above-mentioned phenyl group, dibenzothiophenyl group, phenanthryl group, fluorenyl group, triphenylenyl group, and naphthyl group is exemplified by an alkyl group such as a methyl group, an ethyl group, or a propyl group and an aryl group such as a phenyl group, a fluorenyl group, a phenanthryl group, a triphenylenyl group, or a naphthyl group.

In the formulae [1] to [4], R₁ to R₆ each represent one of a hydrogen atom and an alkyl group having 1 or more to 4 or less carbon atoms.

Examples of the alkyl group represented by each of R₁ to R₆ include a methyl group, an ethyl group, an n-propyl group, an iso-propyl group, an n-butyl group, an iso-butyl group, a sec-butyl group, and a tert-butyl group.

In the formulae [1] to [4], R₁ and R₂ may be identical to or different from each other.

In the formula [3], R₃ and R₄ may be identical to or different from each other.

In the formula [4], R₅ and R₆ may be identical to or different from each other.

The fused polycyclic compound of the present invention may be synthesized, for example, according to a synthesis route shown below.

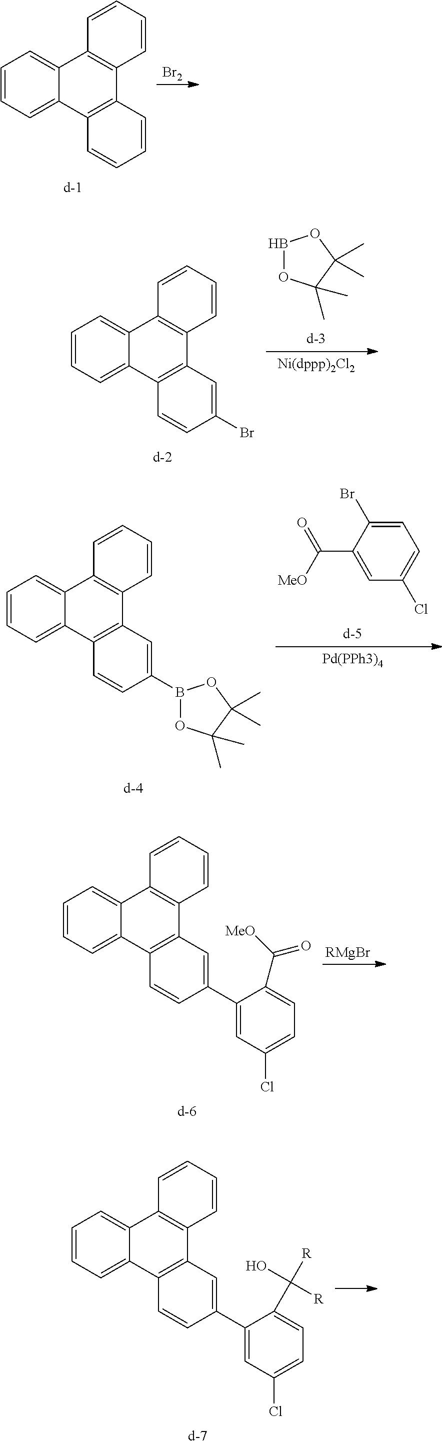

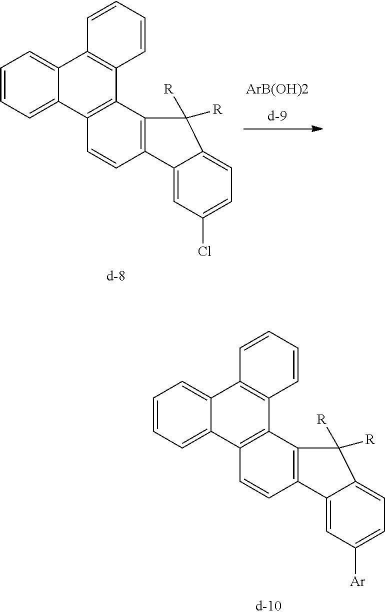

In the above-mentioned synthesis scheme, Compound d-8, one of the intermediates, is a compound having a mother skeleton of the fused polycyclic compound of the present invention. Here, Compound d-8 is synthesized, for example, by the following processes (i) to (v) using triphenylene (Compound d-1) as a starting material.
(i) Bromination of triphenylene (synthesis of Compound d-2)
(ii) Formation of a pinacol boronic acid ester from triphenylene bromide (synthesis of Compound d-4)
(iii) Suzuki-Miyaura coupling reaction of the triphenylenyl boronic acid ester (Compound d-4) synthesized in the process (ii) and methyl bromochlorobenzoate (Compound d-5) (synthesis of Compound d-6)
(iv) Grignard reaction (synthesis of Compound d-7)
(v) Cyclodehydration reaction with polyphosphoric acid (synthesis of Compound d-8)

Meanwhile, Compound d-5 is a compound important in the synthesis of a chloro form (Compound d-8) effective as a raw material for synthesizing each of the compounds represented by the formulae [1] to [4]. It should be noted that an intermediate d-5 shown in the above-mentioned synthesis scheme includes a chlorine atom at the 4-position of a benzene ring, and the chlorine atom may be substituted by any other halogen atom, or the chlorine atom may be substituted by a triflate group or a pinacol boronic acid group.

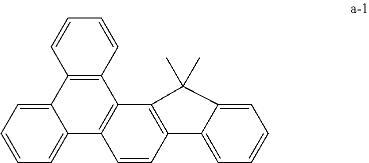 [SMILES: CC1(C)c2ccccc2-c2ccc3c4ccccc4c4ccccc4c3c21]

Next, characteristics of the fused polycyclic compound of the present invention are described. The following compound (a-1) is a compound serving as a mother skeleton of the fused polycyclic compound of the present invention.

As a compound similar to the above-mentioned compound (a-1), there is given the following compound (a-2).

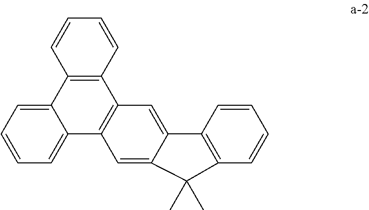 [SMILES: CC1(C)c2ccccc2-c2cc3c4ccccc4c4ccccc4c3cc21]

Here, the compound (a-1) has low molecular association property as compared to the compound (a-2). The compound (a-1) and the compound (a-2) both have a skeleton formed by the fusion of a triphenylene skeleton and a dimethylindene skeleton, but the fusion occurs in different directions with respect to a fluorene ring in both the compounds. As a result, the above-mentioned feature is obtained. That is, in the compound (a-1), which serves as a mother skeleton of the fused polycyclic compound of the present invention, a distance between the triphenylene skeleton, which has strong molecular association property, and each of two methyl groups is closer than that in the compound (a-2). Thus, it can be said that, when molecules of the compound (a-1) are to associate with each other, a methyl group possessed by a predetermined molecule suppresses the stacking of triphenylene skeletons possessed by other molecules, resulting in low molecular association property.

The low molecular association property leads to the suppression of concentration quenching and excimer emission due to molecular association, and hence is advantageous for emission characteristics of a compound.

Next, a difference between substitution positions of a substituent to be introduced into the compound (a-1) and the compound (a-2) is described.

The fused polycyclic compound of the present invention has a feature of including the following compound (a-1) as a mother skeleton and having a substituent introduced at the α-position of the mother skeleton.

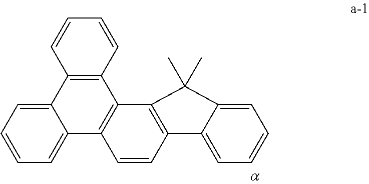 [SMILES: CC1(C)c2ccccc2-c2ccc3c4ccccc4c4ccccc4c3c21]

The above-mentioned feature leads to a feature in that a conjugation formed by a triphenylene ring and a benzene ring does not undergo any further extension via the substituent, i.e., a conjugation possessed by the mother skeleton is broken between the mother skeleton and the substituent. Thus, the lowest triplet excited state energy (T₁ energy) of the fused polycyclic compound of the present invention depends on the mother skeleton (a-1) of the compound, and high T₁ energy is maintained.

On the other hand, a compound including a compound represented by the following structure (a-2) as a mother skeleton and having a substituent at the β-position of the mother skeleton has a feature in that a conjugation formed by a triphenylene ring and a benzene ring undergoes further extension via the substituent.

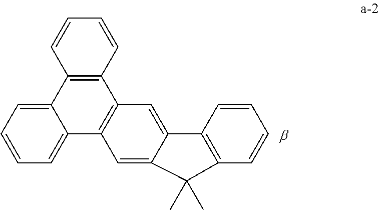 [SMILES: CC1(C)c2ccccc2-c2cc3c4ccccc4c4ccccc4c3cc21]

By virtue of the above-mentioned feature, the lowest triplet excited state energy (T₁ energy) of a-2 depends on an interaction between a-2 and the substituent at the β-position (extended conjugation), and is lower T₁ energy than that of the fused polycyclic compound of the present invention.

Here, the inventors of the present invention measured T₁ energy values of the following compounds in toluene dilute solutions. It should be noted that, in the measurement of T₁, a toluene solution (1×10⁻⁴ mol/l) was cooled to 77 K and measured for its phosphorescence emission spectrum at an excitation wavelength of 350 nm, and the resultant first emission peak was used as T₁. The device used was a spectrophotometer U-3010 manufactured by Hitachi, Ltd.

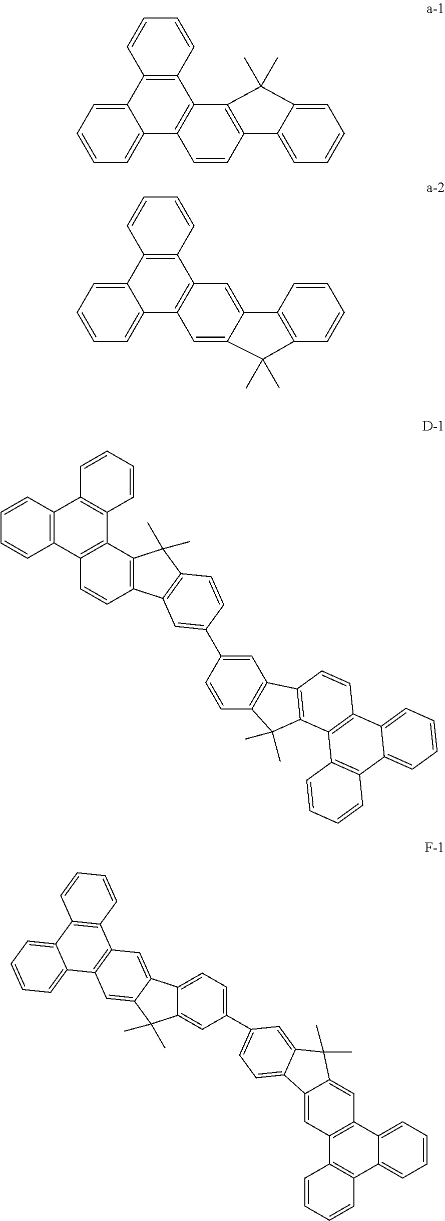

TABLE 1

| Compound | T₁ [nm] |
|---|---|
| a-1 | 482 |
| a-2 | 482 |
| D-1 | 482 |
| F-1 | 529 |

Table 1 shows that T₁ of Compound D-1, which is the fused polycyclic compound of the present invention, is the same as that of its own partial skeleton a-1. This indicates that a conjugation of two skeletons a-1 is broken.

On the other hand, T₁ of Compound F-1, which corresponds to a comparative compound, shifts to the much longer wavelength side than that of its own partial skeleton a-2. This indicates that a conjugation of two skeletons a-2 is maintained.

Meanwhile, Ar shown in each of the formulae [1] to [4] preferably represents an aryl group having high T₁ and is selected from aryl groups each having T₁ of 530 nm or less. Specifically, Ar is selected from benzene, dibenzothiophene, phenanthrene, fluorene, triphenylene, and naphthalene. It should be noted that the aryl group represented by Ar may further have a substituent.

Based on the foregoing, the fused polycyclic compound of the present invention has T₁ ranging from 470 nm or more to 500 nm or less by use of the mother skeleton a-1 having high T₁ and the substitution with the Ar group at a predetermined position.

The fused polycyclic compound of the present invention has the above-mentioned action and effect, and hence can provide a light emitting device having high efficiency when used as a material for an organic light emitting device, in particular, a light emitting material.

Meanwhile, T₁ of a phosphorescent light emitting material which emits green phosphorescent light is 490 nm or more to 530 nm or less, and the fused polycyclic compound of the present invention has higher T₁ energy than that of the phosphorescent light emitting material. Accordingly, the use of the fused polycyclic compound of the present invention as a host or an electron transporting material for a light emitting layer in an organic light emitting device which emits green phosphorescent light can improve the emission efficiency of the element. In this case, a phosphorescent light emitting compound is a guest (phosphorescent light emitting material) for the light emitting layer.

The fused polycyclic compound of the present invention has a feature in that the aryl group represented by Ar or a-1 is bonded to the mother skeleton a-1 at a predetermined position. Here, the planarity of the whole molecule is broken by the bonding of Ar to the mother skeleton a-1, which is effective for forming a stable amorphous film.

Accordingly, the use of the fused polycyclic compound of the present invention as a material for an organic light emitting device can provide a light emitting device having improved durability.

Specific examples of the fused polycyclic compound of the present invention are shown below. In this regard, however, the present invention is by no means limited thereto.

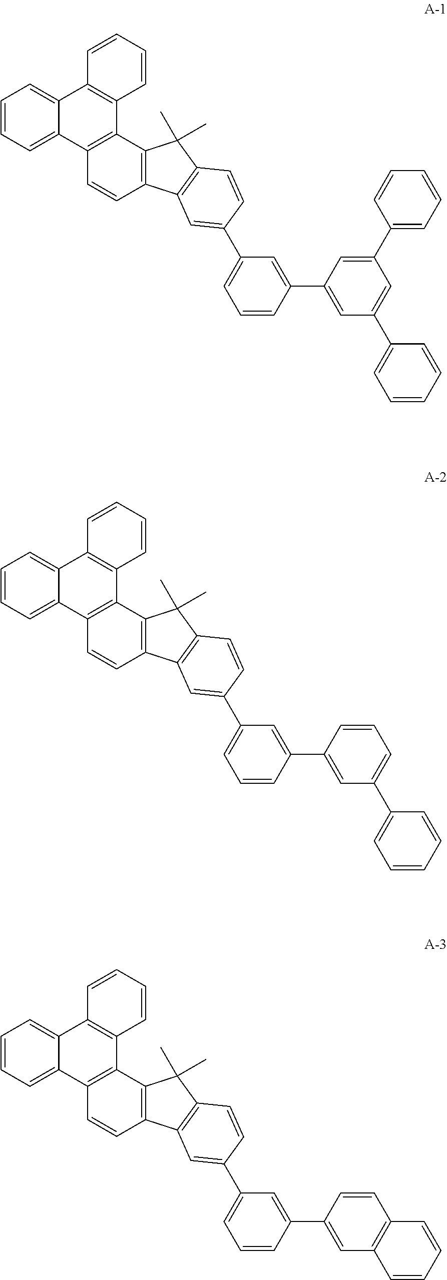

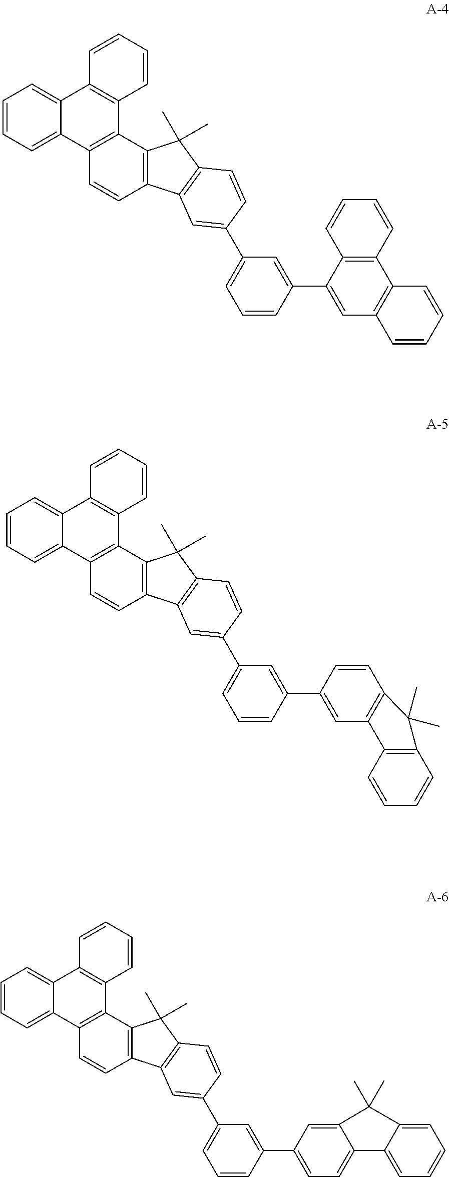

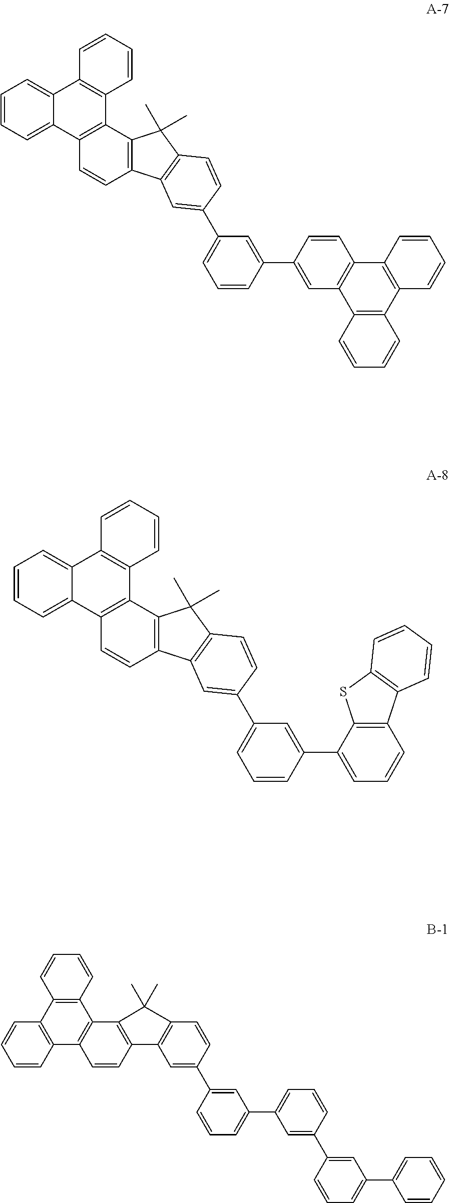

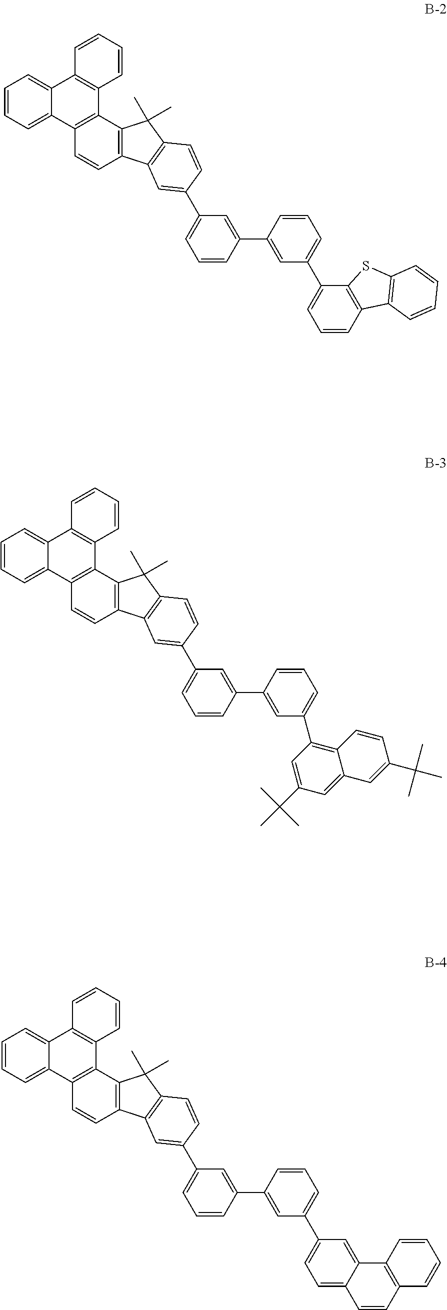

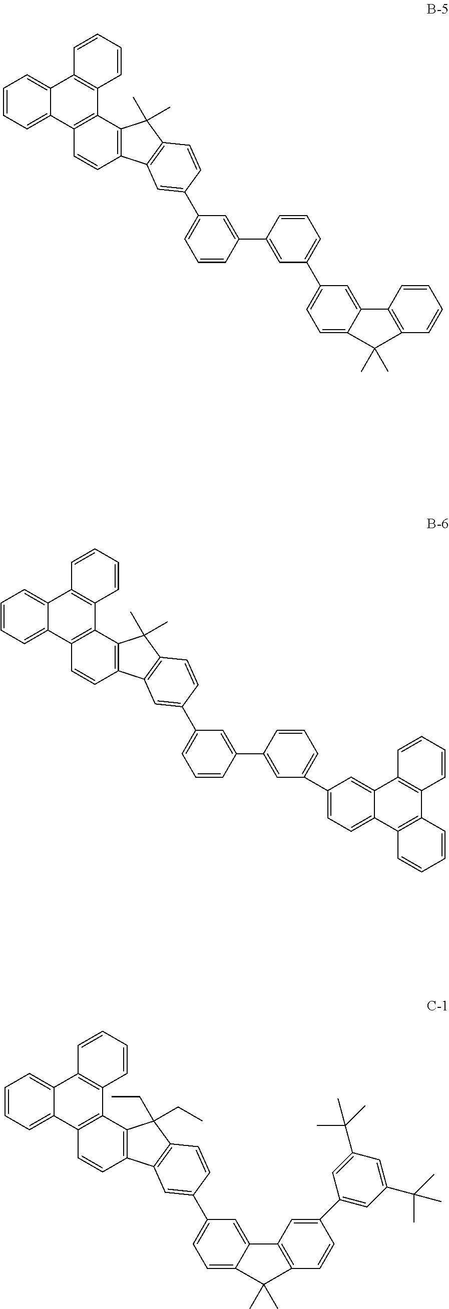

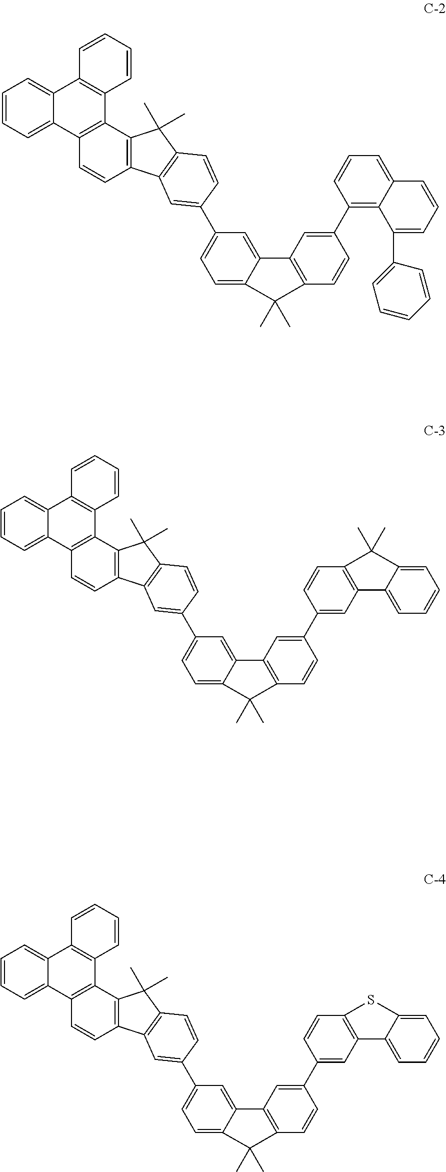

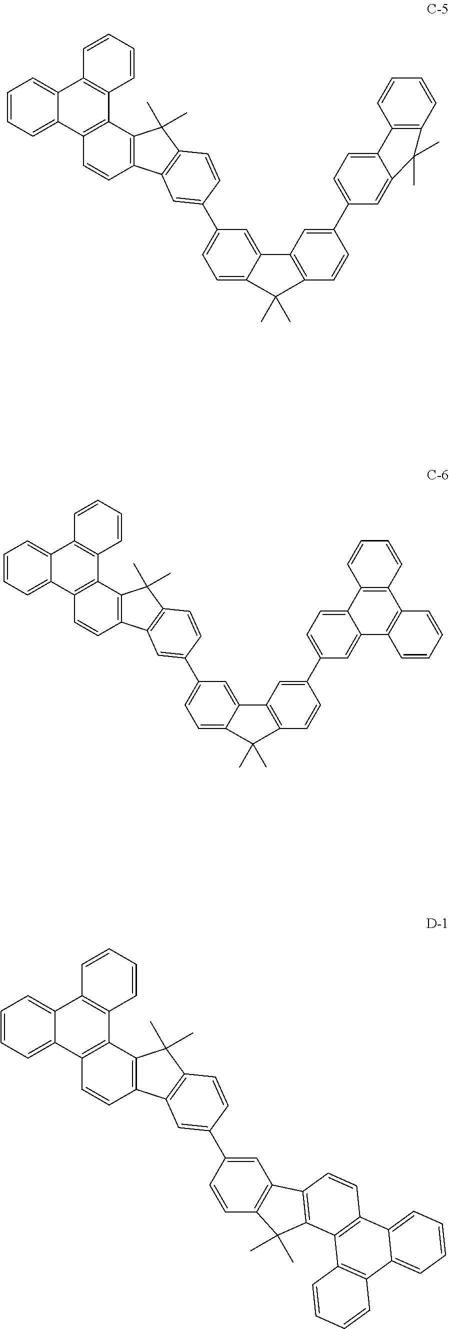

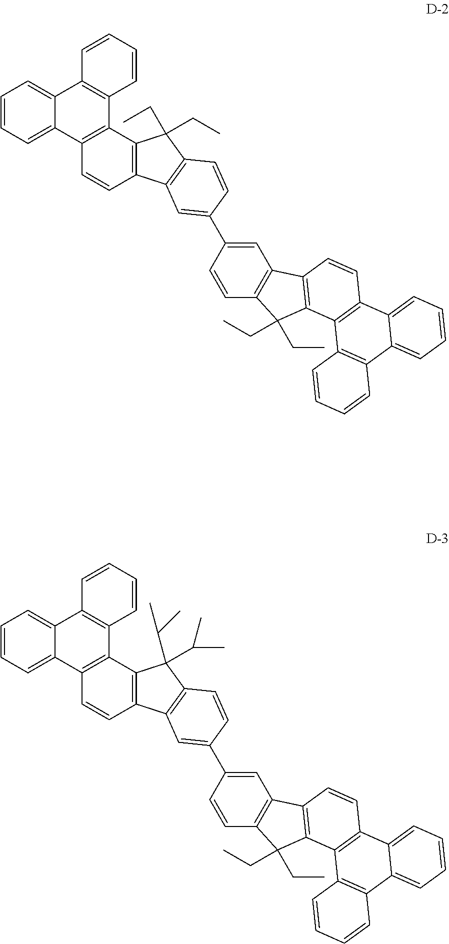

In the above-mentioned specific examples, the compounds belonging to Group A are a group of compounds each represented by the formula [1], i.e., compounds in each of which the mother skeleton (a-1) and the aryl group are linked together via a phenylene group. Here, each of the compounds belonging to Group A has a small molecular weight. Hence, each of the compounds can be formed into a thin film at a lower vapor deposition temperature by vapor deposition.

In the above-mentioned specific examples, the compounds belonging to Group B are a group of compounds each represented by the formula [2], i.e., compounds in each of which the mother skeleton (a-1) and the aryl group are linked together via a biphenylene group. Here, each of the compounds belonging to Group B includes a number of bonds that allow rotation in a molecule. Hence, when each of the compounds is formed into an amorphous film, the film has high stability.

In the above-mentioned specific examples, the compounds belonging to Group C are a group of compounds each represented by the formula [3], i.e., compounds in each of which the mother skeleton (a-1) and the aryl group are linked together via a fluorenylene group. Here, the fluorenylene group, which links the mother skeleton (a-1) and the aryl group together, is rigid. Hence, when each of the compounds is formed into an amorphous film, the film has high electron and hole mobilities.

In the above-mentioned specific examples, the compounds belonging to Group D are a group of compounds each represented by the formula [4], i.e., dimers of the mother skeletons (a-1). Here, each of the compounds belonging to Group D has high molecular symmetry. Hence, when each of the compounds is formed into an amorphous film, the film has high electron and hole mobilities.

Here, it can be said that, out of the compounds shown in the above-mentioned specific examples, compounds in each of which Ar shown in the formulae [1] to [4] represents dibenzothiophene, specifically Compounds A-8, B-2, and C-4 are preferred materials from the viewpoint of having high hole injecting/transporting property.

Next, the organic light emitting device of the present invention is described.

The organic light emitting device of the present invention is constructed of a pair of electrodes, i.e., an anode and a cathode, and an organic compound layer disposed between the anode and the cathode.

In the present invention, an organic compound layer, which is a member for constructing an organic light emitting device, may be a single layer or a laminate formed of multiple layers as long as the organic compound layer includes a light emitting layer or a layer having a light emitting function.

When the organic compound layer is formed of multiple layers, a layer which is a layer other than the light emitting layer (or the layer having a light emitting function) and is included in the organic compound layer is exemplified by a hole injection layer, a hole transport layer, a light emitting layer, a hole blocking layer, an electron transport layer, an electron injection layer, and an exciton blocking layer. As a matter of course, one or more layers may be selected from the above-mentioned group and used in combination.

It should be noted that the construction of the organic light emitting device of the present invention is by no means limited thereto. For example, there may be adopted a variety of layer constructions as described below. That is, an insulating layer, an adhesion layer, or an interference layer may be provided at an interface between each of electrodes and an organic compound layer, or an electron transport layer or a hole transport layer may be constructed of two layers having different ionization potentials.

In the organic light emitting device of the present invention, an embodiment of the element may be the so-called top emission mode involving extracting light from an electrode on the side opposite to the substrate, or may be the so-called bottom emission mode involving extracting light from the substrate side. Alternatively, there may be adopted a construction in which light is extracted from both sides using a substrate and electrodes each formed of a transparent material.

In the organic light emitting device of the present invention, the fused polycyclic compound of the present invention is included in the organic compound layer. In the organic light emitting device of the present invention, the organic compound layer including the fused polycyclic compound of the present invention is not particularly limited, but the fused polycyclic compound is preferably included in the light emitting layer. In the organic light emitting device of the present invention, the light emitting layer may be a layer formed of only the fused polycyclic compound of the present invention, but is preferably a layer formed of a host and a guest.

Here, the fused polycyclic compound of the present invention may be used as the host for the light emitting layer or may be used as the guest, but is preferably used as the host for the light emitting layer. Here, the use of the fused polycyclic compound of the present invention as a host to be used in combination with a guest which emits phosphorescent light is preferred from the viewpoint of emission efficiency. In particular, the use of the fused polycyclic compound of the present invention in combination with a guest which emits green to red light having an emission peak in a region of 490 nm to 660 nm reduces a loss in triplet energy, thereby providing a light emitting device having high efficiency.

It should be noted that, when the fused polycyclic compound of the present invention is used as the guest, the concentration of the guest to the host is preferably 0.1 wt % or more to 30 wt % or less, more preferably 0.5 wt % or more to 10 wt % or less with respect to the total amount of the light emitting layer.

In the organic light emitting device of the present invention, in addition to the fused polycyclic compound of the present invention, as necessary, any other compound may be used as a material for constructing the organic light emitting device. Specifically, a conventionally known low-molecular or high-molecular hole injecting/transporting material, host, guest, or electron injecting/transporting material, or the like may be used in combination with the fused polycyclic compound of the present invention.

Hereinafter, examples of those compounds are given.

The hole injecting/transporting material is preferably a material having a high hole mobility. Low-molecular and high-molecular materials each having hole injecting performance or hole transporting performance are exemplified by, but should not be limited to, a triarylamine derivative, a phenylenediamine derivative, a stilbene derivative, a phthalocyanine derivative, a porphyrin derivative, poly(vinylcarbazole), poly(thiophene), and other conductive polymers.

Examples of the host include, but should not be limited to, a triarylamine derivative, a phenylene derivative, a fused ring aromatic compound (for example, a naphthalene derivative, a phenanthrene derivative, a fluorene derivative, or a chrysene derivative), an organic metal complex (for example, an organic aluminum complex such as tris(8-quinolinolato)aluminum, an organic beryllium complex, an organic iridium complex, or an organic platinum complex), and a polymer derivative such as a poly(phenylenevinylene) derivative, a poly(fluorene) derivative, a poly(phenylene) derivative, a poly(thienylenevinylene) derivative, or a poly(acetylene) derivative.

The guest is preferably a phosphorescent light emitting material. Specific examples thereof include Ir complexes shown below and platinum complexes each having phosphorescent light emitting property.

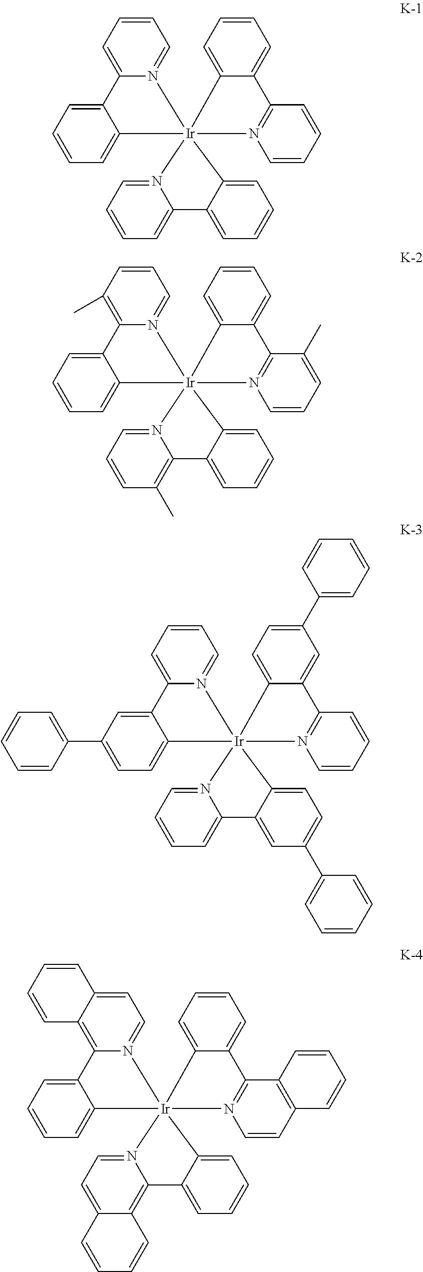

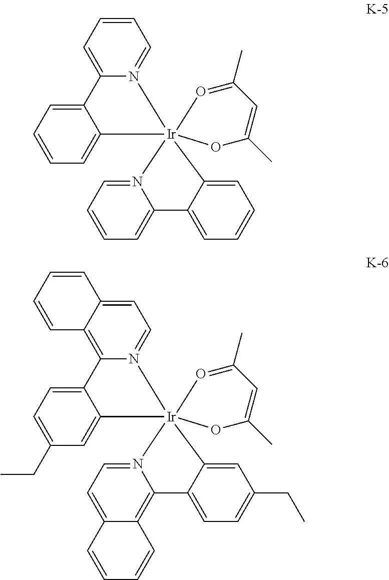

Further, a fluorescent light emitting dopant may also be used, and examples thereof include a fused ring compound (for example, a fluorene derivative, a naphthalene derivative, a pyrene derivative, a perylene derivative, a tetracene derivative, an anthracene derivative, or rubrene), a quinacridone derivative, a coumarin derivative, a stilbene derivative, an organic aluminum complex such as tris(8-quinolinolato)aluminum, an organic beryllium complex, and a polymer derivative such as a poly(phenylenevinylene) derivative, a poly(fluorene) derivative, or a poly(phenylene) derivative.

The electron injecting/transporting material is selected in consideration of, for example, a balance with the hole mobility of the hole injecting material or the hole transporting material. A material having electron injecting performance or electron transporting performance is exemplified by, but should not be limited to, an oxadiazole derivative, an oxazole derivative, a pyrazine derivative, a triazole derivative, a triazine derivative, a quinoline derivative, a quinoxaline derivative, a phenanthroline derivative, and an organic aluminum complex.

It is recommended that a material for constructing an anode have as large a work function as possible. Examples thereof include metal elements such as gold, platinum, silver, copper, nickel, palladium, cobalt, selenium, vanadium, and tungsten, or alloys including combinations of multiple kinds of those metal elements, and metal oxides such as tin oxide, zinc oxide, indium oxide, indium tin oxide (ITO), and indium zinc oxide. Further, conductive polymers such as polyaniline, polypyrrole, and polythiophene may also be used. One kind of those electrode substances may be used alone, or multiple kinds thereof may be used in combination. Further, the anode may be constructed of a single layer or may be constructed of multiple layers.

Meanwhile, it is recommended that a material for constructing a cathode have a small work function. Examples of the material include alkali metals such as lithium, alkaline earth metals such as calcium, and metal elements such as aluminum, titanium, manganese, silver, lead, and chromium. Alternatively, alloys including combinations of multiple kinds of those metal elements may also be used. For example, magnesium-silver, aluminum-lithium, aluminum-magnesium, and the like may be used. Metal oxides such as indium tin oxide (ITO) may also be utilized. One kind of those electrode substances may be used alone, or multiple kinds thereof may be used in combination. Further, the cathode may be constructed of a single layer or may be constructed of multiple layers.

In the organic light emitting device of the present invention, a layer including the fused polycyclic compound of the present invention and any other layer are formed by the following method. In general, a layer is formed by a vacuum vapor deposition method, an ionization vapor deposition method, a sputtering method, or a plasma method. Alternatively, the layer may be formed by dissolving the compound in an appropriate solvent and subjecting the resultant to a known coating method (for example, a spin coating method, a dipping method, a casting method, an LB method, or an ink jet method). Here, when the layer is formed by a vacuum vapor deposition method, a solution coating method, or the like, the layer is hard to undergo crystallization and the like and is excellent in stability over time. Further, when the film is formed by a coating method, the film may also be formed in combination with an appropriate binder resin.

Examples of the above-mentioned binder resin include, but not limited to, a poly(vinylcarbazole) resin, a polycarbonate resin, a polyester resin, an ABS resin, an acrylic resin, a polyimide resin, a phenolic resin, an epoxy resin, a silicone resin, and a urea resin. Further, one kind of those binder resins may be used alone as a homopolymer or copolymer, or two or more kinds thereof may be used as a mixture. In addition, a known additive such as a plasticizer, an antioxidant, or an ultraviolet absorber may be used in combination with the binder resin, as necessary.

The organic light emitting device of the present invention may be used for a display apparatus and lighting equipment. In addition, the element may be used for a light source for exposure of an electrophotographic image forming device, or a backlight of a liquid crystal display apparatus, for example.

The display apparatus includes the organic light emitting device of the present invention in a display unit. The display unit includes multiple pixels. The pixels each include the organic light emitting device according to this embodiment and a TFT element as an example of a switching element for controlling emission luminance, and an anode or a cathode of the organic light emitting device is connected to a drain electrode or a source electrode of the TFT element. The display apparatus may be used as an image display apparatus such as a PC.

The display apparatus includes an image input unit for inputting information from an area CCD, a linear CCD, a memory card, and the like, and may be an image output apparatus for outputting the input image to a display unit. Further, a display unit included in an image pickup device or an ink jet printer may be provided with both of an image output function, which displays an image based on image information input from the outside, and an input function, which serves as an operation panel and inputs processing information for an image. Further, the display apparatus may be used for a display unit of a multifunction printer.

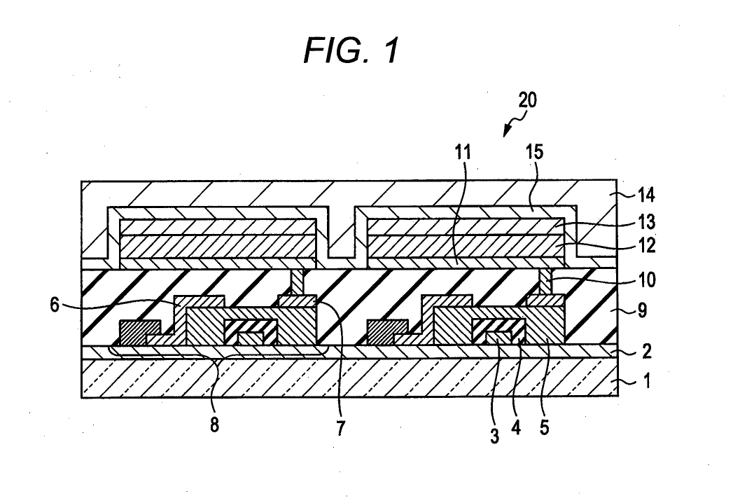

Next, a display apparatus using the organic light emitting device according to this embodiment is described with reference to FIG. 1.

FIG. 1 is a cross-sectional schematic diagram illustrating an example of a display apparatus including the organic light emitting device of the present invention and a TFT element as an example of a switching element electrically connected to the organic light emitting device. Two sets of the organic light emitting device and the TFT element are illustrated in a display apparatus 20 of FIG. 1. Details of the structure are described below.

The display apparatus 20 of FIG. 1 includes a substrate 1 made of glass or the like and a moisture-proof film 2 for protecting a TFT element or an organic compound layer on the substrate. Further, a gate electrode 3 made of metal is represented by reference numeral 3, a gate insulating film 4 is represented by reference numeral 4, and a semiconductor layer is represented by reference numeral 5.

A TFT element 8 includes the semiconductor layer 5, a drain electrode 6, and a source electrode 7. An insulating film 9 is provided above the TFT element 8. An anode 11 of the organic light emitting device is connected to the source electrode 7 via a contact hole 10. The display apparatus is not limited to the above-mentioned construction, and any one of the anode and a cathode has only to be connected to any one of the source electrode and the drain electrode of the TFT element.

It should be noted that, in the display apparatus 20 of FIG. 1, an organic compound layer 12 may be a single organic compound layer or multiple organic compound layers but is illustrated like a single layer. A first protective layer 14 and a second protective layer 15 for suppressing the deterioration of the organic light emitting device are provided above a cathode 13.

In the display apparatus according to this embodiment, a switching element is not particularly limited, and a monocrystalline silicon substrate, an MIM element, an a-Si type element, or the like may be used.

### EXAMPLES

Hereinafter, the present invention is described in detail by way of examples. In this regard, however, the present invention is by no means limited thereto.

### Example 1

### Synthesis of Exemplified Compound A-8

Synthesis was performed according to the following synthesis scheme.

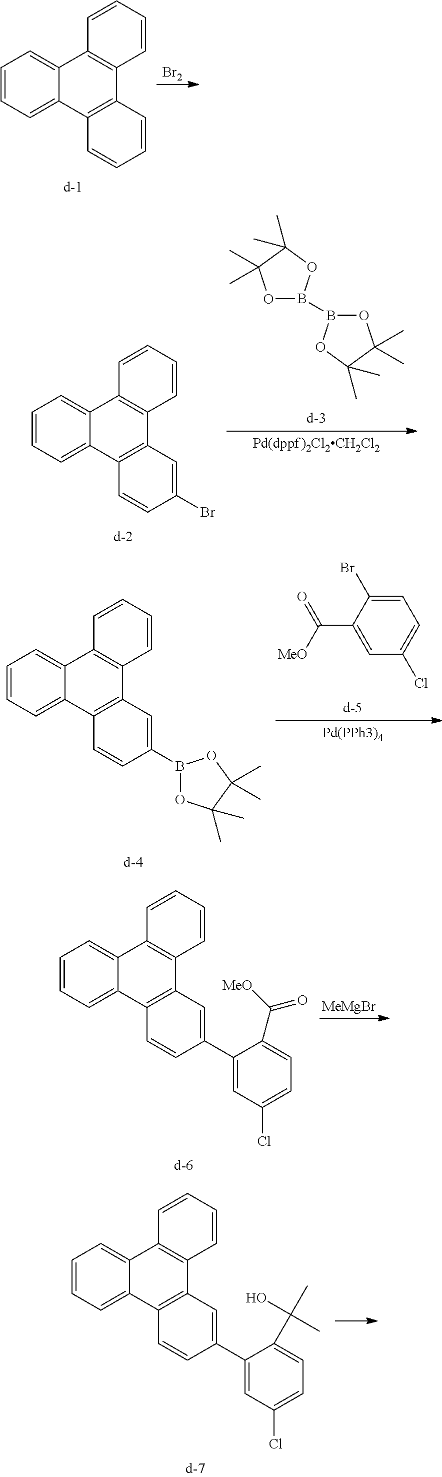

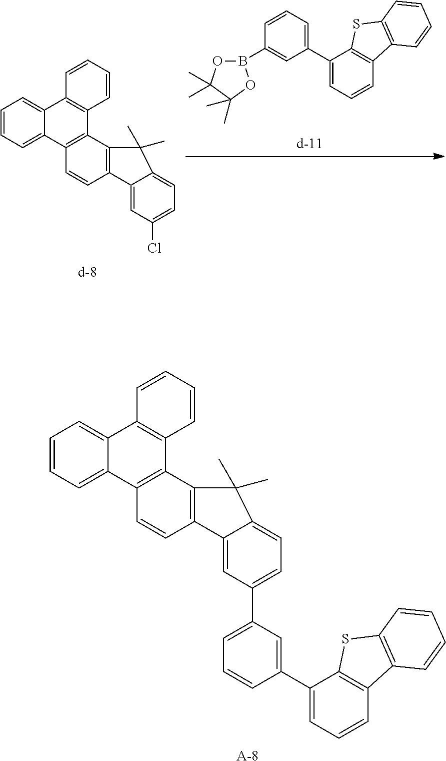 [SMILES: CC1(C)c2ccc(Cl)cc2-c2ccc3c4ccccc4c4ccccc4c3c21 ; CC1(C)OB(c2cccc(-c3cccc4c3sc3ccccc34)c2)OC1(C)C ; CC1(C)c2ccc(-c3cccc(-c4cccc5c4sc4ccccc45)c3)cc2-c2ccc3c4ccccc4c4ccccc4c3c21 ; "A-8"]

### (1) Synthesis of Compound d-2

The following reagent and solvent were loaded into a 500-ml three-necked flask.
Compound d-1: 9.99 g (43.8 mmol)
Dichloromethane: 300 ml

Next, the reaction solution was stirred at room temperature under a nitrogen atmosphere, and to the stirred solution was added dropwise a mixed solution of 7.7 g (48.2 mmol) of bromine and 7.0 ml of dichloromethane. After the dropwise addition of the mixed solution, the reaction solution was stirred at room temperature for 12 hours. After the completion of the reaction, the reaction solution was poured into a solution of sodium thiosulfate. The organic layer was then extracted with chloroform, and the resultant organic layer was dried over anhydrous sodium sulfate. The organic layer was then concentrated under reduced pressure to give a crude product. Next, the resultant crude product was purified by silica gel column chromatography (developing solvent: toluene-heptane mixed solvent) to afford 11.6 g of Compound d-2 as a white solid (yield: 86.3%).

### (2) Synthesis of Compound d-4

A nitrogen atmosphere was established in a 300-ml three-necked flask. After that, the following reagents and solvent were loaded into the flask.
Compound d-2: 11.5 g (37.4 mmol)
Compound d-3: 11.4 g (44.9 mmol)
Potassium acetate: 6.61 g (67.4 mmol)
Dioxane: 100 ml

Next, the reaction solution was stirred at room temperature under a nitrogen atmosphere, and to the stirred solution were added 1.53 g (1.87 mmol) of a bis(diphenylphosphino)ferrocene palladium(II) dichloride dichloromethane adduct. The reaction solution was then warmed to a temperature of 100° C. and stirred at the same temperature (100° C.) for 4 hours. After the completion of the reaction, the solvent in the reaction solution was evaporated under reduced pressure to give a crude product. Next, the resultant crude product was purified by silica gel column chromatography (developing solvent: chloroform-heptane mixed solvent) to afford 10.42 g of Compound d-4 as a white solid (yield: 68.6%).

### (3) Synthesis of Compound d-6

A nitrogen atmosphere was established in a 200-ml three-necked flask. After that, the following reagents and solvents were loaded into the flask.
Compound d-4: 7.08 g (20.0 mmol)
Compound d-5: 5.46 g (22.0 mmol)
Sodium carbonate: 10.6 g (100 mmol)
Toluene: 100 ml
Ethanol: 20 ml
Water: 100 ml

Next, the reaction solution was stirred at room temperature under a nitrogen atmosphere, and to the stirred solution were added 1.16 g of tetrakis(triphenylphosphine)palladium(0). Next, the reaction solution was warmed to a temperature of 80° C. and stirred at the same temperature (80° C.) for 12 hours. After the completion of the reaction, the organic layer was extracted with toluene, and the resultant organic layer was dried over anhydrous sodium sulfate. The organic layer was then concentrated under reduced pressure to give a crude product. Next, the resultant crude product was purified by silica gel column chromatography (developing solvent: toluene-ethyl acetate mixed solvent) to afford 4.92 g of Compound d-6 as a white solid (yield: 62%).

### (4) Synthesis of Compound d-7

The following reagent and solvent were loaded into a 100-ml three-necked flask.
Compound d-6: 3.46 g (9.06 mmol)
THF: 80 ml

Next, the reaction solution was stirred with cooling with ice under a nitrogen atmosphere, and to the stirred solution were gradually added dropwise 22.6 ml of methylmagnesium bromide. After the completion of the dropwise addition, the reaction solution was warmed to room temperature and stirred at the same temperature (room temperature) for 15 hours. The reaction solution was then poured into 100 ml of water. The organic layer was then extracted with toluene, and the resultant organic layer was dried over anhydrous sodium sulfate. The organic layer was then concentrated under reduced pressure to give a crude product. Next, the resultant crude product was purified by silica gel column chromatography (developing solvent: toluene) to afford 2.16 g of Compound d-7 as a white solid (yield: 60.2%).

### (5) Synthesis of Compound d-8

The following reagent and solvents were loaded into a 50-ml three-necked flask.
Compound d-7: 2.10 g (5.30 mmol)
Polyphosphoric acid: 30 ml
Chloroform: 20 ml

Next, the reaction solution was warmed to a temperature of 60° C. and then stirred at the same temperature (60° C.) for 3 hours. The reaction solution was then poured into 30 ml of water. The organic layer was then extracted with toluene, and the resultant organic layer was dried over anhydrous sodium sulfate. The organic layer was then concentrated under reduced pressure to give a crude product. Next, the resultant crude product was purified by silica gel column chromatography (developing solvent: toluene-heptane mixed solvent), and an isomer was then separated and removed by gel filtration chromatography. The above-mentioned processes afford 1.65 g of Compound d-8 as a white solid (yield: 82.2%).

### (6) Synthesis of Exemplified Compound A-8

The following reagents and solvents were loaded into a 50-ml three-necked flask.
Compound d-8: 0.378 g (1.00 mmol)
Compound d-11: 0.425 g (1.10 mmol)
Potassium phosphate: 1.06 g
Toluene: 5 ml
Water: 0.1 ml

Next, the reaction solution was stirred at room temperature under a nitrogen atmosphere, and to the stirred solution were added the following reagents.
Palladium acetate: 22 mg
Compound d-12: 82 mg

Next, the reaction solution was warmed to a temperature of 90° C. and then stirred at the same temperature (90° C.) for 5 hours. After the completion of the reaction, the organic layer was extracted with toluene, and the resultant organic layer was dried over anhydrous sodium sulfate. The organic layer was then concentrated under reduced pressure to give a crude product. Next, the resultant crude product was purified by silica gel column chromatography (developing solvent: toluene-heptane mixed solvent) to afford 0.440 g of Exemplified Compound A-8 as a white solid (yield: 73.1%).

M⁺ (602) of Exemplified Compound A-8 was confirmed by mass spectrometry.

Next, T₁ of Exemplified Compound A-8 in a toluene dilute solution was measured. Specifically, a toluene solution (1×10⁻⁴ mol/l) was cooled to 77 K, the toluene solution was irradiated with light at an excitation wavelength of 350 nm to measure a phosphorescence emission spectrum, and the first emission peak obtained by the measurement was used as T₁. It should be noted that, in the measurement, the device used was a spectrophotometer U-3010 manufactured by Hitachi, Ltd. As a result of the measurement, T₁ of Exemplified Compound A-8 was found to be 482 nm. Further, Exemplified Compound A-8 was measured for its ionization potential. Specifically, a deposition film having a thickness of 20 nm formed on a glass substrate by a vacuum vapor deposition method was measured for its ionization potential using an atmospheric photoelectron spectrometer (AC-3 manufactured by RIKEN KEIKI CO., LTD.). As a result of the measurement, the ionization potential was found to be 6.16 eV.

### Example 2

### Synthesis of Exemplified Compound A-1

Exemplified Compound A-1 was synthesized by the same method as in Example 1 except that Compound e-1 shown below was used in place of Compound d-11 in Example 1(6).

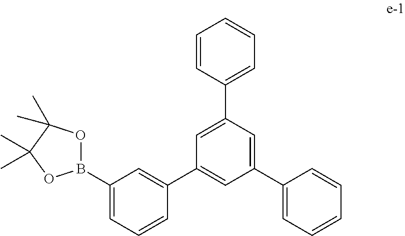 [SMILES: CC1(C)OB(c2cccc(-c3cc(-c4ccccc4)cc(-c4ccccc4)c3)c2)OC1(C)C]

M⁺ (648) of Exemplified Compound A-1 was confirmed by mass spectrometry. Further, T₁ of Exemplified Compound A-1 in a toluene dilute solution was measured by the same method as in Example 1, and T₁ was found to be 481 nm.

### Example 3

### Synthesis of Exemplified Compound A-5

Exemplified Compound A-5 was synthesized by the same method as in Example 1 except that Compound e-2 shown below was used in place of Compound d-11 in Example 1(6).

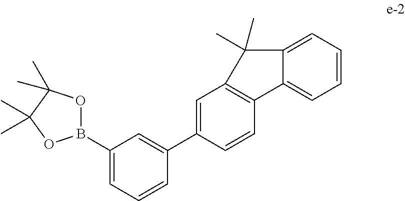 [SMILES: CC1(C)c2ccccc2-c2ccc(-c3cccc(B4OC(C)(C)C(C)(C)O4)c3)cc21]

M⁺ (612) of Exemplified Compound A-5 was confirmed by mass spectrometry. Further, T₁ of Exemplified Compound A-5 in a toluene dilute solution was measured by the same method as in Example 1, and T₁ was found to be 482 nm.

### Example 4

### Synthesis of Exemplified Compound B-2

Exemplified Compound B-2 was synthesized by the same method as in Example 1 except that Compound e-3 shown below was used in place of Compound d-11 in Example 1(6).

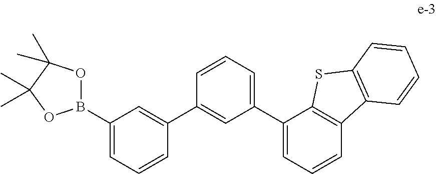 [SMILES: CC1(C)OB(c2cccc(-c3cccc(-c4cccc5c4sc4ccccc45)c3)c2)OC1(C)C]

M⁺ (678) of Exemplified Compound B-2 was confirmed by mass spectrometry. Further, T₁ of Exemplified Compound B-2 in a toluene dilute solution was measured by the same method as in Example 1, and T₁ was found to be 481 nm.

### Example 5

### Synthesis of Exemplified Compound B-5

Exemplified Compound B-5 was synthesized by the same method as in Example 1 except that Compound e-4 shown below was used in place of Compound d-11 in Example 1(6).

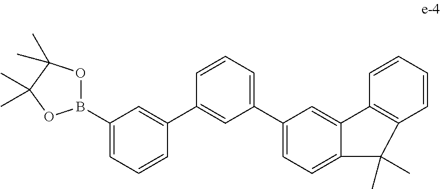 [SMILES: CC1(C)c2ccccc2-c2cc(-c3cccc(-c4cccc(B5OC(C)(C)C(C)(C)O5)c4)c3)ccc21]

M⁺ (688) of Exemplified Compound B-5 was confirmed by mass spectrometry. Further, T₁ of Exemplified Compound B-5 in a toluene dilute solution was measured by the same method as in Example 1, and T₁ was found to be 482 nm.

### Example 6

### Synthesis of Exemplified Compound B-6

Exemplified Compound B-6 was synthesized by the same method as in Example 1 except that Compound e-5 shown below was used in place of Compound d-11 in Example 1(6).

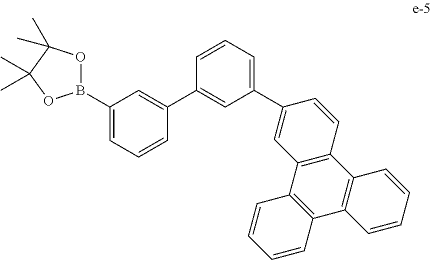 [SMILES: CC1(C)OB(c2cccc(-c3cccc(-c4ccc5c6ccccc6c6ccccc6c5c4)c3)c2)OC1(C)C]

M⁺ (722) of Exemplified Compound B-6 was confirmed by mass spectrometry. Further, T₁ of Exemplified Compound B-6 in a toluene dilute solution was measured by the same method as in Example 1, and T₁ was found to be 482 nm.

### Example 7

### Synthesis of Exemplified Compound C-3

Exemplified Compound C-3 was synthesized by the same method as in Example 1 except that Compound e-6 shown below was used in place of Compound d-11 in Example 1(6).

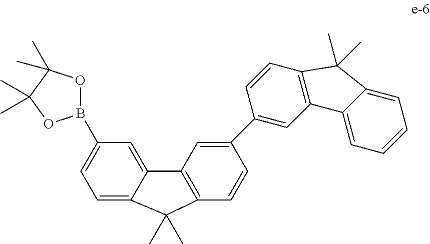 [SMILES: CC1(C)c2ccccc2-c2cc(-c3ccc4c(c3)-c3cc(B5OC(C)(C)C(C)(C)O5)ccc3C4(C)C)ccc21]

M⁺ (728) of Exemplified Compound C-3 was confirmed by mass spectrometry. Further, T₁ of Exemplified Compound C-3 in a toluene dilute solution was measured by the same method as in Example 1, and T₁ was found to be 483 nm.

### Example 8

### Synthesis of Exemplified Compound D-1

Exemplified Compound D-1 was synthesized according to a synthesis scheme shown below.

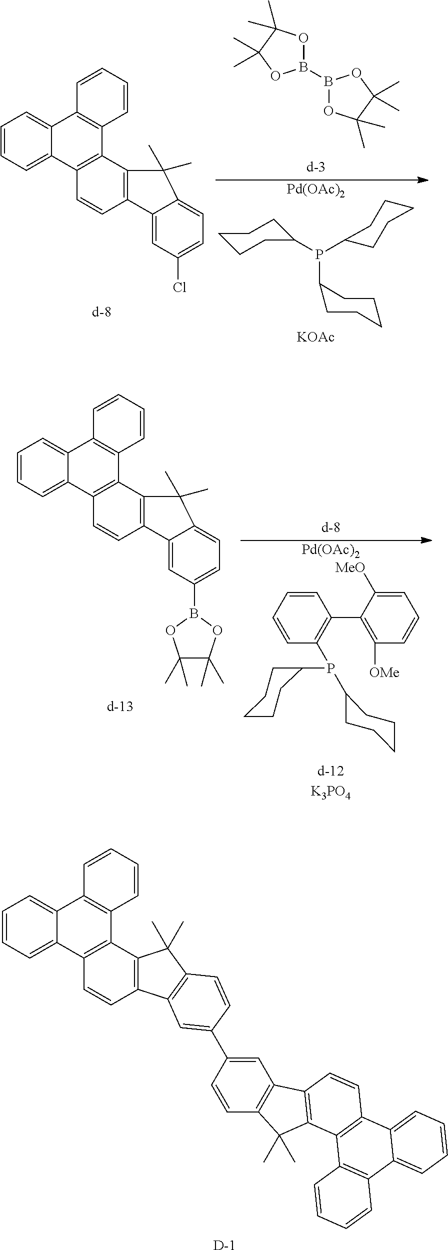

### (1) Synthesis of Compound d-13

A nitrogen atmosphere was established in a 100-ml three-necked flask. After that, the following reagents and solvent were loaded into the flask.
Compound d-8: 0.378 g (1.00 mmol)
Compound d-3: 0.305 g (1.20 mmol)
Potassium acetate: 0.294 g (3.00 mmol)
Dioxane: 30 ml

Next, the reaction solution was stirred at room temperature under a nitrogen atmosphere, and to the stirred solution were added the following reagents.
Palladium acetate: 22 mg
Tricyclohexylphosphine: 56 mg

Next, the reaction solution was warmed to a temperature of 100° C. and then stirred at the same temperature (100° C.) for 6 hours. After the completion of the reaction, the solvent in the reaction solution was evaporated under reduced pressure to give a crude product. Next, the resultant crude product was purified by silica gel column chromatography (developing solvent: chloroform-heptane mixed solvent) to afford 0.446 g of Compound d-13 as a white solid (yield: 65.0%).

### (2) Synthesis of Exemplified Compound D-1

The following reagents and solvents were loaded into a 50-ml three-necked flask.
Compound d-13: 0.400 g (0.85 mmol)
Compound d-8: 0.302 g (0.80 mmol)
Potassium phosphate: 1.0 g
Toluene: 5 ml
Water: 0.1 ml

Next, the reaction solution was stirred at room temperature under a nitrogen atmosphere, and to the stirred solution were added the following reagents.
Palladium acetate: 22 mg
Compound d-12: 82 mg

Next, the reaction solution was heated to a temperature of 90° C. and then stirred at the same temperature (90° C.) for 5 hours. After the completion of the reaction, the organic layer was extracted with toluene, and the resultant organic layer was dried over anhydrous sodium sulfate. The organic layer was then concentrated under reduced pressure to give a crude product. Next, the resultant crude product was purified by silica gel column chromatography (developing solvent: toluene-heptane mixed solvent) to afford 0.390 g of Exemplified Compound D-1 as a white solid (yield: 72.0%).

M⁺ (686) of Exemplified Compound D-1 was confirmed by mass spectrometry. Further, T₁ of Exemplified Compound D-1 in a toluene dilute solution was measured by the same method as in Example 1, and T₁ was found to be 482 nm.

### Comparative Example 1

### Synthesis of Comparative Compound F-1

Comparative Compound F-1 shown below was synthesized by the same method as in Example 8 except that Compound e-15 shown below was used in place of Compound d-8 in Examples 8 (1) and 8(2).

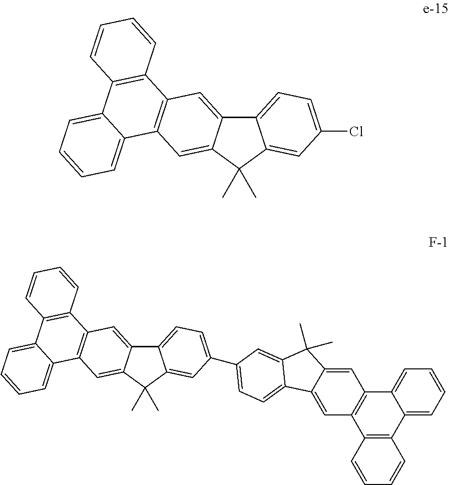

It should be noted that Compound e-15 is obtained, for example, by performing synthesis by the same method as in Examples 1(1) to 1(5) except that Compound e-14 shown below is used in place of Compound d-5 in Example 1(3).

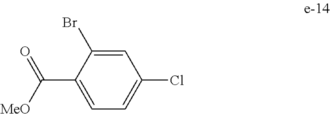 [SMILES: COC(=O)c1ccc(Cl)cc1Br]

M⁺ (686) of Comparative Compound F-1 was confirmed by mass spectrometry. Further, T₁ of Comparative Compound F-1 in a toluene dilute solution was measured by the same method as in Example 1, and T₁ was found to be 529 nm.

### Example 9

An organic light emitting device having the construction of “anode/hole transport layer/light emitting layer/electron transport layer/cathode” successively provided on a glass substrate (substrate) was produced by the following method. Some of materials used in this example are shown below.

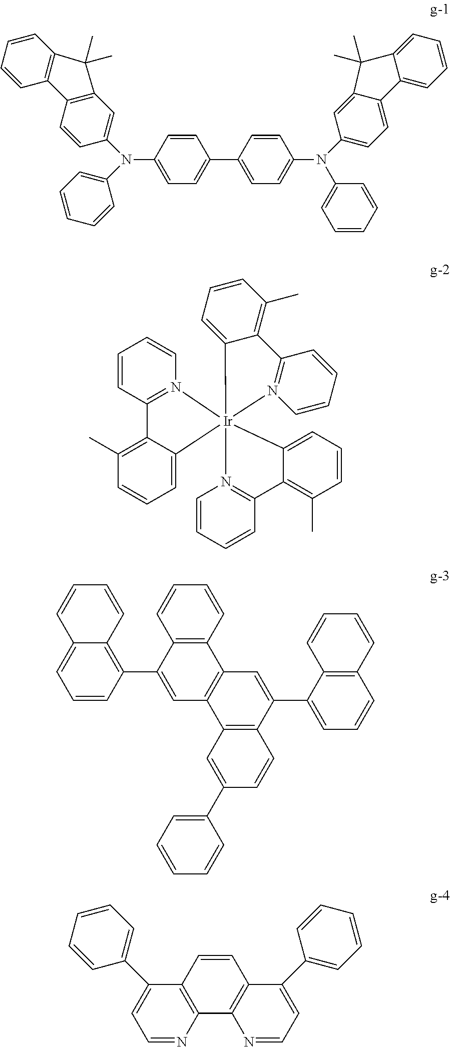

ITO was formed into a film to serve as an anode on a glass substrate by a sputtering method. In this case, the thickness of the anode was set to 120 nm. The substrate having formed thereon the ITO electrode as described above was used as a transparent conductive supporting substrate (substrate with an ITO electrode) in the following steps.

Next, organic compound layers and electrode layers shown in Table 2 below were continuously formed as films on the substrate with the ITO electrode by vacuum vapor deposition through resistance heating in a vacuum chamber at 1×10⁻⁵ Pa. In this case, an opposite electrode was produced so as to have an area of 3 mm².

TABLE 2

| | Material | Thickness [nm] |
|---|---|---|
| Hole transport layer | g-1 | 30 |
| Light emitting layer | Host: A-8 | 30 |
| | Guest: g-2 | |
| | (host:guest = 85:15 | |
| | (weight ratio)) | |
| Hole-exciton | g-3 | 10 |
| blocking layer | | |
| Electron transport | g-4 | 100 |
| layer | | |
| First metal | LiF | 1 |
| electrode layer | | |
| (cathode) | | |
| Second metal | Al | 30 |
| electrode layer | | |
| (cathode) | | |

A voltage of 4.0 V was applied to the resultant organic light emitting device while the ITO electrode was used as a positive electrode and the Al electrode was used as a negative electrode. As a result, the current density was 3.40 mA/cm². Further, the voltage in the case where the emission luminance of the element was set to 4,000 cd/m² was 4.2 V. In addition, the element was observed to emit green light having an emission efficiency of 66 cd/A and CIE chromaticity coordinates of (0.35, 0.62).

In addition, the organic light emitting device of this example was continuously driven while the current density was kept at 40 mA/cm² under a nitrogen atmosphere. As a result, the time period until the luminance becomes half of the initial luminance was 80 hours or more.

### Example 10

An organic light emitting device was produced by the same method as in Example 9 except that Exemplified Compound A-1 was used in place of Exemplified Compound A-8 as the host included in the light emitting layer in Example 9.

A voltage was applied to the organic light emitting device produced in this example while the ITO electrode was used as a positive electrode and the Al electrode was used as a negative electrode. As a result, the voltage at an emission luminance of 4,000 cd/m² was 4.3 V. Further, the element was observed to emit green light having an emission efficiency of 63 cd/A and CIE chromaticity coordinates of (0.35, 0.62).

### Example 11

An organic light emitting device was produced by the same method as in Example 9 except that Exemplified Compound A-5 was used in place of Exemplified Compound A-8 as the host included in the light emitting layer in Example 9.

A voltage was applied to the organic light emitting device produced in this example while the ITO electrode was used as a positive electrode and the Al electrode was used as a negative electrode. As a result, the voltage at an emission luminance of 4,000 cd/m² was 4.3 V. Further, the element was observed to emit green light having an emission efficiency of 60 cd/A and CIE chromaticity coordinates of (0.35, 0.62).

### REFERENCE SIGNS LIST

1 substrate
2 moisture-proof film
3 gate electrode
4 gate insulating film
5 semiconductor layer
6 drain electrode
7 source electrode
8 TFT element
9 insulating film
10 contact hole
11 anode
12 organic compound layer
13 cathode
14 first protective layer
15 second protective layer
20 display apparatus

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-286970, filed Dec. 24, 2010, which is hereby incorporated by reference herein in its entirety.

1. A fused polycyclic compound, which is represented by any one of the following general formulae [1] to [4]: 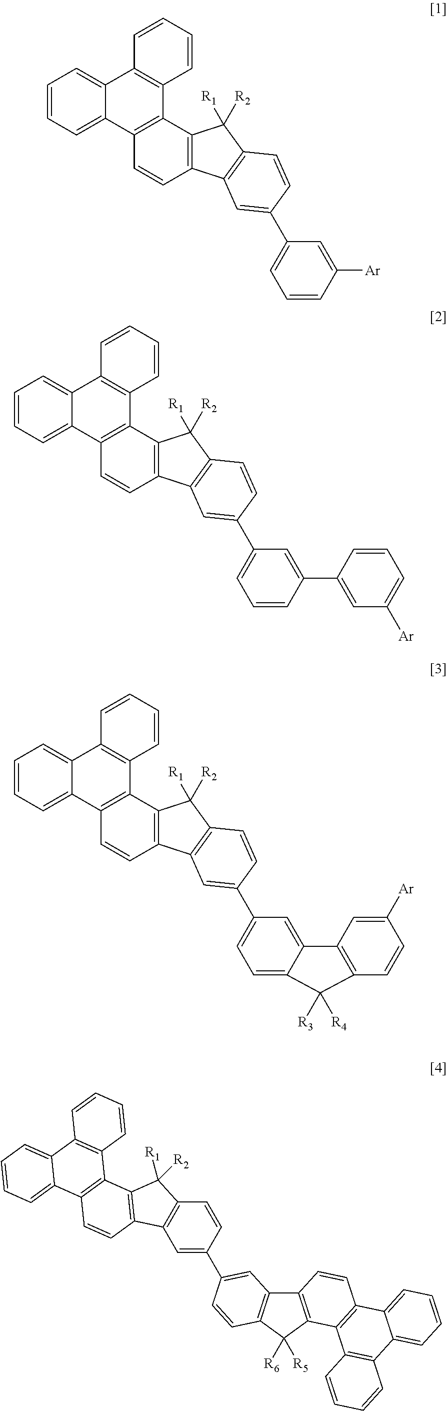
in the formulae [1] to [4], Ar represents one of a substituted or unsubstituted phenyl group, a substituted or unsubstituted dibenzothiophenyl group, a substituted or unsubstituted phenanthryl group, a substituted or unsubstituted fluorenyl group, a substituted or unsubstituted triphenylenyl group, and a substituted or unsubstituted naphthyl group, and R₁ to R₆ each represent one of a hydrogen atom and an alkyl group having 1 or more to 4 or less carbon atoms; in the formulae [1] to [4], R₁ and R₂ may be identical to or different from each other; in the formula [3], R₃ and R₄ may be identical to or different from each other; and in the formula [4], R₅ and R₆ may be identical to or different from each other.

2. The fused polycyclic compound according to claim 1, wherein the Ar represents substituted or unsubstituted dibenzothiophene.

3. An organic light emitting device comprising:
an anode;
a cathode; and
an organic compound layer disposed between the anode and the cathode,
wherein the organic compound layer comprises the fused polycyclic compound according to claim 1.

4. The organic light emitting device according to claim 3, wherein:
the fused polycyclic compound is contained in a light emitting layer;
the light emitting layer comprises a host and a guest; and
the host comprises the fused polycyclic compound.

5. The organic light emitting device according to claim 4, wherein the guest comprises a phosphorescent light emitting material.

6. A display apparatus comprising:
the organic light emitting device according to claim 3; and
a switching element electrically connected to the organic light emitting device.

7. An image output apparatus comprising:
a display unit for displaying an image; and
an input unit for inputting information,
wherein:
the display unit comprises multiple pixels; and
the pixels each comprise the organic light emitting device according to claim 3 and a switching element connected to the organic light emitting device.

8. A display apparatus comprising:
the organic light emitting device according to claim 4; and
a switching element electrically connected to the organic light emitting device.

9. An image output apparatus comprising:
a display unit for displaying an image; and
an input unit for inputting information,
wherein:
the display unit comprises multiple pixels; and
the pixels each comprise the organic light emitting device according to claim 4 and a switching element connected to the organic light emitting device.

10. A display apparatus comprising:
the organic light emitting device according to claim 5; and
a switching element electrically connected to the organic light emitting device.

11. An image output apparatus comprising:
a display unit for displaying an image; and
an input unit for inputting information,
wherein:
the display unit comprises multiple pixels; and
the pixels each comprise the organic light emitting device according to claim 5 and a switching element connected to the organic light emitting device.

12. A lighting equipment comprising the organic light emitting device according to claim 3.

13. A lighting equipment comprising the organic light emitting device according to claim 4.

14. A lighting equipment comprising the organic light emitting device according to claim 5.
